(19) United States
(12) Patent Application Publication
(10) Pub. No.: US 2023/0270687 A1
(43) Pub. Date: Aug. 31, 2023

(54) HOMEOPATHIC ANTIVIRAL HYDROGEL PATCHES

(71) Applicant: JSIC Laboratories, LLC, Dana Point, CA (US)

(72) Inventor: Ryan Goerig, Dana Point, CA (US)

(21) Appl. No.: 18/165,394

(22) Filed: Feb. 7, 2023

Related U.S. Application Data
(60) Provisional application No. 63/313,982, filed on Feb. 25, 2022.

(51) Int. Cl. A61K 9/70; A61K 36/71; A61K 41/00

(52) U.S. Cl. CPC A61K 9/7069; A61K 36/71; A61K 9/7053; A61K 41/0004

(57) ABSTRACT

Described herein are examples of patches which include a hydrogel having an active homeopathic ingredient dissolved therein. The active homeopathic ingredient Berberis vulgaris is dissolved in the hydrogel and is effective at treating molluscum contagiosum. The patches are cured and shaped so that the active ingredient kills the virus over time when the patch is in contact with an infected area. The patches temporarily adhere to the skin and are effective at curing and preventing the spread of viral skin infection, both to other people and to other parts of the infected person's body. The patches may be packaged and sold as an over-the-counter homeopathic product.

### CROSS-REFERENCE TO RELATED APPLICATIONS

The present application claims the benefit of U.S. Provisional Patent Application No. 63/313,982 entitled “HOMEOPATHIC ANTIVIRAL HYDROGEL PATCHES”, filed on Feb. 25, 2022. The entire contents of the above-listed application are hereby incorporated by reference for all purposes.

### BACKGROUND

Viral skin conditions affect large numbers of people around the world each year. Such conditions can be highly contagious, uncomfortable, or may cause blemishes on the skin. In particular, the condition molluscum contagiosum affects an estimated 6 million people in the United States. There exists a need for treatments of molluscum contagiosum and other viral skin conditions that are effective at curing and preventing spread of the conditions.

### BRIEF DESCRIPTION OF THE DRAWINGS

The present description will be understood more fully when viewed in conjunction with the accompanying drawings of various examples of homeopathic antiviral hydrogel patches. The description is not meant to limit the homeopathic antiviral hydrogel patches to the specific examples. Rather, the specific examples depicted and described are provided for explanation and understanding of homeopathic antiviral hydrogel patches. Throughout the description the drawings may be referred to as drawings, figures, and/or FIGs.

FIG. 1 illustrates a homeopathic antiviral hydrogel patch, according to an embodiment.

FIG. 2 illustrates a package containing a plurality of homeopathic antiviral hydrogel patches, according to an embodiment.

FIG. 3 illustrates a side-by-side comparison of an affected area before, during, and after treatment, according to an embodiment.

FIG. 4 illustrates an annotated side-by-side comparison of an affected area before, during, and after treatment according to an embodiment.

FIG. 5 illustrates a method for treating molluscum contagiosum, according to an embodiment.

### DETAILED DESCRIPTION

Homeopathic antiviral hydrogel patches as disclosed herein will become better understood through a review of the following detailed description in conjunction with the figures. The detailed description and figures provide merely examples of the various embodiments of homeopathic antiviral hydrogel patches. Many variations are contemplated for different applications and design considerations; however, for the sake of brevity and clarity, all the contemplated variations may not be individually described in the following detailed description. Those skilled in the art will understand how the disclosed examples may be varied, modified, and altered and not depart in substance from the scope of the examples described herein.

A conventional treatment for a viral skin condition may include topical creams or liquids that are applied to the affected area. However, such treatments may not be FDA-approved over-the-counter homeopathic products. For molluscum contagiosum in particular, no Food and Drug Administration (“FDA”) approved treatment currently exists. Non-FDA approved topicals exist, but do not prevent the spread of the virus causing the condition.

Topical products such as Zymaderm, Tretinoin, Imiquimod, and Condylox have not been shown to have better than about a 10-40% cure rate after twelve weeks of treatment and are not FDA-approved for treatment of molluscum contagiosum. In office treatments exist, such as cryotherapy or laser surgery, but these treatments can cause pain or discomfort, may cause scarring or dyspigmentation, and are often less convenient for patients than at-home treatments.

Homeopathic products can be FDA approved and used over-the counter but must be indicated for a specific use. There are over 500 botanical homeopathic active ingredients listed in the Homeopathic Pharmacopoeia of the United States (“HPUS”). Traditionally, none of these ingredients have been known to be effective at treating molluscum contagiosum.

Implementations of the homeopathic antiviral hydrogel patches described herein may address some or all of the problems described above. In particular, the homeopathic ingredient Berberis vulgaris, common name barberry, has been found by the inventors to be highly effective for treating molluscum contagiosum when dissolved in a hydrogel patch and applied to the skin as an over-the-counter treatment according to embodiments of the invention disclosed herein

By dissolving a homeopathic active ingredient that is effective at treating molluscum contagiosum in a hydrogel patch that is applicable to the skin, the patches disclosed herein may both simultaneously treat the virus while preventing spread of the virus, both to other people and to other parts of the infected person's body (autoinoculation) and may be manufactured as an over-the-counter product.

The hydrogel can be selected from excipients, including, but not limited to, polyethylene oxide, polyvinyl alcohol, hydroxypropyl methylcellulose, carbomers (carbopol), polyvinylpyrrolidone (PVP), glycerin, sodium acrylate, and/or other cellulose polymers, polyethylene oxide. Examples of suitable, commercially available polyethylene oxide polymers include Poly Ox®, WSRN-1105 and/or WSR coagulant, available from Dow Chemical. Additionally suitable excipients for use as the hydrogel can include ethyl cellulose, cellulose acetate, cellulose acetate propionate, cellulose acetate butyrate, cellulose acetate phthalate, cellulose triacetate, cellulose ether, cellulose ester, cellulose ester ether, acrylic resins comprising copolymers synthesized from acrylic and methacrylic acid esters, the acrylic polymer may be selected from the group consisting of acrylic acid and methacrylic acid copolymers, methyl methacrylate copolymers, ethoxyethylmethacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), glycidyl methacrylate copolymers, hydroxypropyl methyl cellulose, and combinations thereof. Examples of suitable, commercially available hydroxypropyl methylcellulose polymers include Methocel K100 LV and Methocel K4M, available from Dow chemicals.

Implementations of the homeopathic antiviral patches include an active homeopathic ingredient that is dissolved in a hydrogel and applied to an affected area. The patches may be manufactured in a current Good Manufacturing Practices (“cGMP”) facility according to FDA, Federal Trade Commission (“FTC”), and Homeopathic Pharmacopoeia of the United States (“HPUS”) standards for over-the-counter drugs, and are effective at treating and preventing the spread of molluscum contagiosum without requiring a doctor's visit.

The structure and packaging of the pharmaceutical patch can be prepared in accordance with methods and techniques known to persons skilled in the art. The primary components are the backing layer, the adhesive-drug layer (or adhesive-drug matrix), and the release liner.

The backing layer may be comprised of polymeric films such as polyester (PET) or polyethylene (PE) films that support the adhesive drug matrix and protect the transdermal delivery device from the environment. The preferred thickness range for the backing film is from about 2-5 mils (1 mil equals 1/1000 of an inch), and the most preferred thickness range of the backing layer is from about 3-4 mils thick.

The adhesive in the adhesive-drug layer may be a pressure sensitive adhesive (PSA). Useful PSAs in transdermal delivery systems include, but are not limited to, polyisobutylenes (PIB), silicone polymers, and acrylate copolymers, such as acrylic pressure sensitive adhesives, including Duro-Tak 87-2516, 87-2852 and 87-2194, manufactured by Henkel Adhesives. Pills are elastomeric polymers that are commonly used in PSAs, both as primary-base polymers and as tackifiers. PIBs are homopolymers of isobutylene and feature a regular structure of a carbon-hydrogen backbone with only terminal unsaturation. PIBs are marketed under the trade name Oppanol by BASF. The silicone polymers are a high molecular weight polydimethylsiloxane that contains residual silanol functionality (SiOH) on the ends of the polymer chains. Silicone PSAs for use in pharmaceutical applications are available from Dow Corning Corporation, for example under the trade name of BIO-PSA. The release liner can be manufactured in the desired size for the present invention. The release liner may be comprised of silicone (silicon coated release liner) or fluoro-polymer coated polyester film. The release liner protects the pharmaceutical patch during storage and is removed before its use. Silicone-coated release liners are manufactured by Mylan Corporation, Loparex Corporation, and 3M's Drug Delivery Systems. The fluoro-polymer coated release liners are manufactured and supplied by 3M's Drug Delivery Systems and Loparex. The preferred thickness of the release liner is about 2-10 mils, and most preferably about 3-5 mils. Alternatively, the hydrogel can also act as the adhesive.

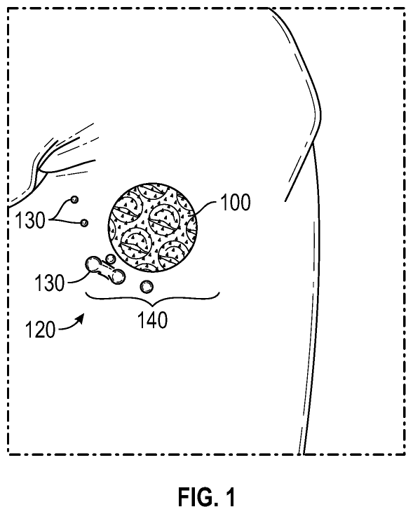

FIG. 1 illustrates a homeopathic antiviral hydrogel patch 100, according to an embodiment. The patch 100 may be applied to the skin 120 as shown. The patch 100 adheres to the skin 120 and remains in place for approximately one week before a new patch 100 is reapplied. The patch comprises a hydrogel medium in which a homeopathic active antiviral ingredient is dissolved.

In the illustrated embodiment, the hydrogel patch 100 is round and approximately 0.5 to 1 inch in diameter, and preferably 0.75 (¾) inch in diameter. The hydrogel is chosen such that the active ingredient is readily dissolvable, so that it may be distributed in the hydrogel and applied in an even concentration over an area of skin 120.

In one embodiment, the hydrogel pad or patch 100 is embedded with a 1% solution of the active homeopathic pharmaceutical ingredient Berberis vulgaris 6×, which has been found through experimentation to be surprisingly effective among the over 500 botanical homeopathic active ingredients listed in the HPUS and can be produced in a lab according to cGMP guidelines and accordance with HPUS guidelines. These sizes and concentrations have been found suitable for treating affected areas of molluscum contagiosum. However, it will be understood by a person of ordinary skill in the art that a range of patch sizes and shapes or active ingredient concentrations may be suitable without departing from the scope of this disclosure. Further, the patches may include various design elements, logos, or coloration on the surface or surfaces of the patch.

In an embodiment a package of pharmaceutical patches can be prepared with 48 pharmaceutical patches provided as four sets of 12 patches. The pharmaceutical patches can include a homeopathic active agent, such as Berberis vulgaris, a carrier, such as a hydrogel, and a patch material prepared from pharmaceutical grade materials. The pharmaceutical patch can include a permeable underlayer and an impermeable top layer. Embodiments of the pharmaceutical patch can include a 1% solution of the Berberis vulgaris dissolved in the hydrogel. Embodiments include the permeable underlayer of the patch material evenly coated with the 1% solution of Berberis vulgaris dissolved in the hydrogel and distributed across the underside of the patch. The impermeable top layer of the pharmaceutical patch can be configured to block transmission of fluids and semi-fluids. Embodiments of the pharmaceutical patch can be cured and shaped. The curing and shaping is configured to prevent exposure of a virus to other and/or prevent self-infection. Embodiments of the pharmaceutical patch can be a rounded (a rounded pharmaceutical patch) with a diameter between 0.5 inches and 1 inch, preferably 0.75 inches. Alternative shapes and size of the patch that provide adequate drug therapy and protection from infection (to other and self-infection) can be prepared according to the present description.

When used to treat molluscum contagiosum, a hydrogel patch 100 is placed on each bump 130 or area affected 140 by the virus. The patch 100 is pressed firmly in place for about 10 seconds and may remain in place for approximately 4-7 days and then replaced until the infection is gone. The patch 100 is cured and shaped so that it effectively seals the virus from exposure. The hydrogel patch's homeopathic ingredient has antiviral properties that kill the virus, and the patch 100 effectively contains and prevents the virus' spread during treatment, thus effectively curing and containing the infection within eight to twelve weeks. This treatment course has been found effective for curing molluscum contagiosum. However, a longer or shorter treatment course, or a treatment course involving replacing the patches 100 more or less often would be understood by a person of ordinary skill in the art to fall within the scope of the present invention.

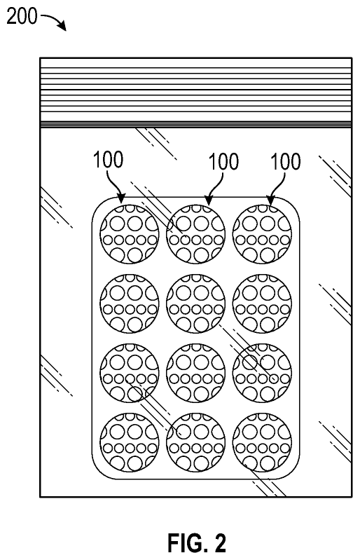

FIG. 2 illustrates a package 200 containing a plurality of homeopathic antiviral hydrogel patches 100, according to an embodiment. A molluscum contagiosum typically expresses itself as bumps 130 on the skin 120 which average twelve in number. A typical treatment course may last from 8-12 weeks. Accordingly, the patches 100 may be packed and sold as shown in four sets of twelve patches. Such packaging is convenient for a weekly course of treatment for molluscum contagiosum, although the patches may be packaged in greater or smaller number or otherwise packaged.

Embodiments of the package can include package further includes a product insert that instructs a user to: apply a first pharmaceutical patch to an area of infection, wherein the area of infection has been infected with molluscum contagiosum, press the first pharmaceutical patch firmly in place over the area of infection for 10 seconds; allow the first pharmaceutical patch to remain on the area of infection for 4 to 7 days; and replace the first pharmaceutical patch with a second pharmaceutical patch every 4 to 7 days for eight to twelve weeks.

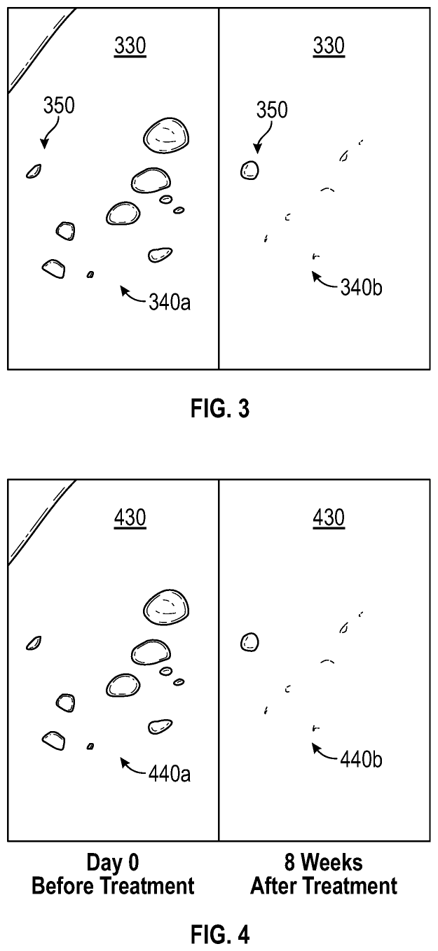

FIG. 3 illustrates a side-by-side comparison of an affected area 330 before, and after treatment, according to an embodiment. The left-most image illustrates a molluscum contagiosum infection prior to treatment 340a. The right-most image illustrates a molluscum contagiosum infection after less than nine weeks of treatment 340b. In each image, the left-most infected spot 350 has been left untreated as a control, demonstrating the effectiveness of the treatment.

FIG. 4 illustrates an annotated side-by-side comparison of an affected area 430 before and after treatment, showing the number of days of treatment and identifying a control, according to an embodiment. As shown, the left-most image illustrates a molluscum contagiosum infection on day one 440a, prior to treatment. The right-most image illustrates a molluscum contagiosum infection on day fifty-six 440b, after eight weeks of treatment. In each image, the left-most infected spot 450 has been left untreated as a control and is indicated by an arrow, demonstrating the effectiveness of the treatment.

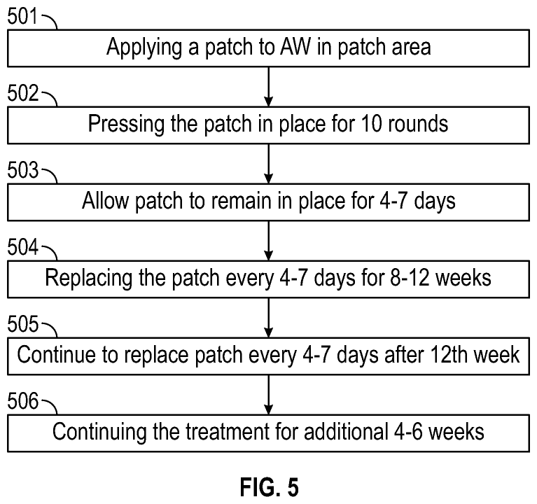

FIG. 5 illustrates a method for treating molluscum contagiosum, according to an embodiment. As illustrated in FIG. 5 embodiments of the topical treatment for molluscum contagiosum can include: step one (501) applying a first pharmaceutical patch to an area of infection, where the area of infection has been infected with molluscum contagiosum; step two (502) pressing the first pharmaceutical patch firmly in place over the area of infection for 10 seconds; step three (503) allowing the first pharmaceutical patch to remain on the area of infection for 4 to 7 days; and step 4 (504) replacing the first pharmaceutical patch with a second pharmaceutical patch every 4 to 7 days for eight to twelve weeks; step five (505) continuing to replace the pharmaceutical patch every 4 to 7 days after the twelfth week if the infection continues, and step six (506) continuing the topical treatment for an additional 4-6 weeks. Embodiments can include step one A (501A) applying the first pharmaceutical patch directly to bumps caused by molluscum contagiosum to seal the viral infection underneath the pharmaceutical patch, step one B (501B) applying the first pharmaceutical patch directly to all visible bumps caused by molluscum contagiosum to prevent infection of others and to prevent self-infection, and step six B (506B) continuing treatment until the infection is no longer visible, cured, or cannot be found by testing.

A feature illustrated in one of the figures may be the same as or similar to a feature illustrated in another of the figures. Similarly, a feature described in connection with one of the figures may be the same as or similar to a feature described in connection with another of the figures. The same or similar features may be noted by the same or similar reference characters unless expressly described otherwise. Additionally, the description of a particular figure may refer to a feature not shown in the particular figure. The feature may be illustrated in and/or further described in connection with another figure.

Elements of processes (i.e. methods) described herein may be executed in one or more ways such as by a human, by a processing device, by mechanisms operating automatically or under human control, and so forth. Additionally, although various elements of a process may be depicted in the figures in a particular order, the elements of the process may be performed in one or more different orders without departing from the substance and spirit of the disclosure herein.

The foregoing description sets forth numerous specific details such as examples of specific systems, components, methods and so forth, in order to provide a good understanding of several implementations. It will be apparent to one skilled in the art, however, that at least some implementations may be practiced without these specific details. In other instances, well-known components or methods are not described in detail or are presented in simple block diagram format in order to avoid unnecessarily obscuring the present implementations. Thus, the specific details set forth above are merely exemplary. Particular implementations may vary from these exemplary details and still be contemplated to be within the scope of the present implementations.

Related elements in the examples and/or embodiments described herein may be identical, similar, or dissimilar in different examples. For the sake of brevity and clarity, related elements may not be redundantly explained. Instead, the use of a same, similar, and/or related element names and/or reference characters may cue the reader that an element with a given name and/or associated reference character may be similar to another related element with the same, similar, and/or related element name and/or reference character in an example explained elsewhere herein. Elements specific to a given example may be described regarding that particular example. A person having ordinary skill in the art will understand that a given element need not be the same and/or similar to the specific portrayal of a related element in any given figure or example in order to share features of the related element.

It is to be understood that the foregoing description is intended to be illustrative and not restrictive. Many other implementations will be apparent to those of skill in the art upon reading and understanding the above description. The scope of the present implementations should, therefore, be determined with reference to the appended claims, along with the full scope of equivalents to which such claims are entitled.

The foregoing disclosure encompasses multiple distinct examples with independent utility. While these examples have been disclosed in a particular form, the specific examples disclosed and illustrated above are not to be considered in a limiting sense as numerous variations are possible. The subject matter disclosed herein includes novel and non-obvious combinations and sub-combinations of the various elements, features, functions and/or properties disclosed above both explicitly and inherently. Where the disclosure or subsequently filed claims recite “a” element, “a first” element, or any such equivalent term, the disclosure or claims is to be understood to incorporate one or more such elements, neither requiring nor excluding two or more of such elements.

As used herein “same” means sharing all features and “similar” means sharing a substantial number of features or sharing materially important features even if a substantial number of features are not shared. As used herein “may” should be interpreted in a permissive sense and should not be interpreted in an indefinite sense. Additionally, use of “is” regarding examples, elements, and/or features should be interpreted to be definite only regarding a specific example and should not be interpreted as definite regarding every example. Furthermore, references to “the disclosure” and/or “this disclosure” refer to the entirety of the writings of this document and the entirety of the accompanying illustrations, which extends to all the writings of each subsection of this document, including the Title, Background, Brief description of the Drawings, Detailed Description, Claims, Abstract, and any other document and/or resource incorporated herein by reference.

As used herein regarding a list, “and” forms a group inclusive of all the listed elements. For example, an example described as including A, B, C, and D is an example that includes A, includes B, includes C, and also includes D. As used herein regarding a list, “or” forms a list of elements, any of which may be included. For example, an example described as including A, B, C, or D is an example that includes any of the elements A, B, C, and D. Unless otherwise stated, an example including a list of alternatively-inclusive elements does not preclude other examples that include various combinations of some or all of the alternatively-inclusive elements. An example described using a list of alternatively-inclusive elements includes at least one element of the listed elements. However, an example described using a list of alternatively-inclusive elements does not preclude another example that includes all of the listed elements. And, an example described using a list of alternatively-inclusive elements does not preclude another example that includes a combination of some of the listed elements. As used herein regarding a list, “and/or” forms a list of elements inclusive alone or in any combination. For example, an example described as including A, B, C, and/or D is an example that may include: A alone; A and B; A, B and C; A, B, C, and D; and so forth. The bounds of an “and/or” list are defined by the complete set of combinations and permutations for the list.

Where multiples of a particular element are shown in a FIG., and where it is clear that the element is duplicated throughout the FIG., only one label may be provided for the element, despite multiple instances of the element being present in the FIG. Accordingly, other instances in the FIG. of the element having identical or similar structure and/or function may not have been redundantly labeled. A person having ordinary skill in the art will recognize based on the disclosure herein redundant and/or duplicated elements of the same FIG. Despite this, redundant labeling may be included where helpful in clarifying the structure of the depicted examples.

The Applicant(s) reserves the right to submit claims directed to combinations and sub-combinations of the disclosed examples that are believed to be novel and non-obvious. Examples embodied in other combinations and sub-combinations of features, functions, elements and/or properties may be claimed through amendment of those claims or presentation of new claims in the present application or in a related application. Such amended or new claims, whether they are directed to the same example or a different example and whether they are different, broader, narrower or equal in scope to the original claims, are to be considered within the subject matter of the examples described herein.

1. A device, comprising:
a package of pharmaceutical patches comprising:
48 pharmaceutical patches provided as four sets of 12 patches; and
the pharmaceutical patches further comprising:
a homeopathic active agent consisting of Berberis vulgaris,
a carrier comprising a hydrogel, and
a patch material prepared from pharmaceutical grade materials, wherein the patch comprises a permeable underlayer and an impermeable top layer;
wherein
the pharmaceutical patch comprises:
a 1% solution of the Berberis vulgaris dissolved in the hydrogel,
the permeable underlayer of the patch material is configured to be coated with the 1% solution of Berberis vulgaris dissolved in the hydrogel, and
the impermeable top layer of the pharmaceutical patch is configured to block transmission of fluids and semi-fluids.

2. The device of claim 1, wherein the pharmaceutical patch is cured and shaped, and wherein the curing and shaping is configured to prevent exposure of a virus.

3. The device of claim 1, wherein the pharmaceutical patch is a rounded pharmaceutical patch with a diameter between 0.5 inches and 1 inch.

4. The device of claim 1, wherein the package is configured to includes a product insert, wherein the product insert is configured to instruct a user to:
apply a first pharmaceutical patch to an area of infection, wherein the area of infection has been infected with molluscum contagiosum,
press the first pharmaceutical patch firmly in place over the area of infection for 10 seconds;
allow the first pharmaceutical patch to remain on the area of infection for 4 to 7 days; and
replace the first pharmaceutical patch with a second pharmaceutical patch every 4 to 7 days for eight to twelve weeks.

5. The device of claim 2, wherein
the pharmaceutical patch is configured to:
prevent transmission of molluscum contagiosum to others, and
to prevent self-infection.

6. The device of claim 1, wherein
the pharmaceutical patch is:
prepared according to Good Manufacturing Practices,
prepared Homeopathic Pharmacopoeia of the United States standards for over-the-counter drugs, and
is an FDA-approved over-the-counter product.

7. The device of claim 1, wherein
the hydrogel is selected from the group consisting of polyethylene oxide, polyvinyl alcohol, hydroxypropyl methylcellulose, carbomers, polyvinylpyrrolidone, cellulose polymers, polyethylene oxide, ethyl cellulose, cellulose acetate, cellulose acetate propionate, cellulose acetate butyrate, cellulose acetate phthalate, cellulose triacetate, cellulose ether, cellulose ester, cellulose ester ether, acrylic resins comprising copolymers synthesized from acrylic and methacrylic acid esters, acrylic acid and methacrylic acid copolymers, methyl methacrylate copolymers, ethoxyethylmethacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), glycidyl methacrylate copolymers, hydroxypropyl methyl cellulose and combinations thereof.

8. The device of claim 1, wherein
the patch further comprises:
an adhesive-drug layer comprising the hydrogel and the Berberis vulgaris,
a backing layer, and
a release liner, and
wherein the release liner is silicon coated.

9. A method, comprising:
a topical treatment for molluscum contagiosum comprising:
applying a first pharmaceutical patch to an area of infection, wherein the area of infection has been infected with molluscum contagiosum;
pressing the first pharmaceutical patch firmly in place over the area of infection for 10 seconds;
allowing the first pharmaceutical patch to remain on the area of infection for 4 to 7 days;
replacing the first pharmaceutical patch with a second pharmaceutical patch every 4 to 7 days for eight to twelve weeks; and
wherein
the pharmaceutical patch comprises:
a homeopathic active agent consisting of Berberis vulgaris,
a carrier comprising a hydrogel, and
a patch material prepared from pharmaceutical grade materials, wherein the patch comprises a permeable underlayer and an impermeable top layer;
wherein
the pharmaceutical patch comprises:
a 1% solution of the Berberis vulgaris dissolved in the hydrogel,
the permeable underlayer of the patch material is coated with the 1% solution of Berberis vulgaris dissolved in the hydrogel, and
the impermeable top layer of the pharmaceutical patch is configured to block transmission of fluids and semi-fluids.

10. The method of claim 9, further comprising:
continuing to replace the pharmaceutical patch every 4 to 7 days after week twelve if the infection continues, and
continuing the topical treatment for an additional 4-6 weeks.

11. The method of claim 9, further comprising applying the first pharmaceutical patch directly to bumps caused by molluscum contagiosum to seal a viral infection underneath the pharmaceutical patch.

12. The method of claim 11, further comprising applying the first pharmaceutical patch directly to all visible bumps caused by molluscum contagiosum to prevent infection of others and to prevent self-infection.

13. The method of claim 10, wherein
the topical treatment is continued until the infection is no longer visible.

14. The method of claim 13, wherein
the pharmaceutical patch further comprises a release liner, a backing layer, and an adhesive-drug layer.

15. The method of claim 14, wherein
the release liner is a silicon coated release liner,
the backing layer is a polymeric film selected from polyester or polyethylene, and
the adhesive-drug layer is the hydrogel and the Berberis vulgaris.

16. A device, comprising:
a pharmaceutical patch comprising:
a homeopathic active agent consisting of Berberis vulgaris,
a carrier comprising a hydrogel, and
a patch material prepared from pharmaceutical grade materials, wherein the patch comprises a permeable underlayer and an impermeable top layer;
wherein
the pharmaceutical patch comprises:
a 1% solution of the Berberis vulgaris dissolved in the hydrogel,
the permeable underlayer of the patch material is configured to be coated with the 1% solution of Berber's vulgaris dissolved in the hydrogel, and
the impermeable top layer of the pharmaceutical patch is configured to block transmission of fluids and semi-fluids.

17. The device of claim 16, wherein
the pharmaceutical patch is:
cured and shaped,
the curing and shaping is configured to prevent exposure of a virus, and
a rounded pharmaceutical patch with a diameter of 0.75 inches.

18. The device of claim 16, wherein the pharmaceutical patch further comprises:
an adhesive-drug layer comprising the hydrogel and the Berberis vulgaris,
wherein the hydrogel is selected from the group consisting of polyethylene oxide, polyvinyl alcohol, hydroxypropyl methylcellulose, carbomers, polyvinylpyrrolidone, cellulose polymers, polyethylene oxide, ethyl cellulose, cellulose acetate, cellulose acetate propionate, cellulose acetate butyrate, cellulose acetate phthalate, cellulose triacetate, cellulose ether, cellulose ester, cellulose ester ether, acrylic resins comprising copolymers synthesized from acrylic and methacrylic acid esters, acrylic acid and methacrylic acid copolymers, methyl methacrylate copolymers, ethoxyethylmethacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), glycidyl methacrylate copolymers, hydroxypropyl methyl cellulose and combinations thereof;
a backing layer; and
a release liner.

19. The device of claim 18, wherein
the release liner is a silicon coated release liner with a thickness between 2 and 10 mils, and
the backing layer is a polymeric film selected from polyester or polyethylene.

20. The device of claim 19, wherein
the adhesive in the adhesive-drug layer is a pressure sensitive adhesive selected from the group consisting of polyisobutylenes, silicone polymers, and acrylate copolymers.
